(19) United States
(12) Patent Application Publication
(10) Pub. No.: US 2024/0254531 A1
(43) Pub. Date: Aug. 1, 2024

(54) Enhancing Coupled Catalytic Activity of Multi-Enzyme Cascades With Liquid-Liquid Phase Separation Using Peptide-Based Condensates

(71) Applicant: The Government of the United States of America, as represented by the Secretary of the Navy, Arlington, VA (US)

(72) Inventors: Joyce C. Breger, Washington, DC (US); Kimihiro Susumu, Washington, DC (US); Igor L. Medintz, Springfield, VA (US); Gregory A. Ellis, Washington, DC (US); Sebastian A. Diaz, Washington, DC (US); Shelby L. Hooe, Washington, DC (US)

(21) Appl. No.: 18/425,004

(22) Filed: Jan. 29, 2024

Related U.S. Application Data
(60) Provisional application No. 63/482,369, filed on Jan. 31, 2023.

(51) Int. Cl. C12P 19/32; C12N 9/02; C12N 9/12; C12N 9/88

(52) U.S. Cl. CPC C12P 19/32; C12N 9/0008; C12N 9/1217; C12N 9/88; C12Y 102/01011; C12Y 207/02004; C12Y 403/01001

(57) ABSTRACT

Enzyme cascade reactions, wherein the product of a first enzyme is the substrate of a second enzyme and so forth, were found to be enhanced by the presence of peptides. It is believed that the peptides operate by forming coacervates, which are membrane-less compartments where, in the case of coacervates formed by peptides, the liquid-liquid phase separation involves water as the continuous phase both inside and outside the coacervate.

### CROSS-REFERENCE TO RELATED APPLICATIONS

This application claims the benefit of U.S. Provisional Patent Application No. 63/482,369 filed Jan. 31, 2023, the entirety of which, including appendix, is incorporated herein by reference. This application is also related to U.S. Pat. No. 11,512,305.

### FEDERALLY-SPONSORED RESEARCH AND DEVELOPMENT

The United States Government has ownership rights in this invention. Licensing inquiries may be directed to Office of Technology Transfer, US Naval Research Laboratory, Code 1004, Washington, DC 20375, USA; +1.202.767.7230; techtran@nrl.navy.mil, referencing NC 211424.

### INCORPORATION BY REFERENCE

This Application incorporates by reference the Sequence Listing XML file submitted herewith via the patent office electronic filing system having the file name “211424 sequences.xml” and created on Jan. 25, 2024 with a file size of 5,252 bytes.

### BACKGROUND

The burgeoning field of synthetic biology seeks to replace many classic wet chemistry industrial processes with greener, biologically-based reactions that are almost all catalyzed by enzymes. There are currently two main approaches being implemented towards this goal, namely that of cell-based and cell-free synthetic biology. Cell-based technologies seek to engineer chassis organisms such as E. coli and other cell types to recombinantly host heterologous genes comprising a designer synthetic or catabolic pathway that produces a desired product in significant quantities from a common substrate. Potentially limiting issues here include energy-intensive maintenance of the cultures, the need to import and export key chemicals/substrates/products across cellular membranes, competing metabolic pathways, and a general intolerance via cellular toxicity to any non-natural substrates and products. Cell-free synthetic biology looks to mitigate these specific issues by utilizing cellular extracts or designer reconstituted versions thereof to perform the required reactions. Interest is also growing in ‘minimalist’ versions of a cell-free synthetic format where reactions only contain the minimal number of components required for a given multistep enzymatic pathway; these typically consist of just the necessary enzymes, cofactors, and substrates.

A need exists for improvements in conducing cell-free enzymatic cascade reactions.

### BRIEF SUMMARY

In one embodiment, a method of conducting an enzyme cascade reaction includes providing a plurality of enzymes configured as an enzymatic cascade wherein the product of a first enzyme is the substrate of a second enzyme and so forth, wherein the enzymatic cascade comprises at least two different enzymes; contacting the enzymatic cascade with a substrate of the first enzyme while contacting the enzymatic cascade with a peptide under conditions effective to form a peptide-driven coacervate; and allowing a reaction to proceed so that each of the plurality of enzymes acts in succession to produce an end product, wherein at least one of the enzymes comprises multiple polyhistidine tags acting to cross-link the nanoparticles into the cluster.

### BRIEF DESCRIPTION OF THE DRAWINGS

FIG. 1 shows data for an enzyme cascade reaction performed without blocking peptides, either as free enzymes or bound to quantum dots (QD). Here and in the other figures, separate assembly indicates that each enzyme was assembly onto the QD individually, prior to the addition of blocking peptide (where present) and then subsequent mixing of the three enzymes.

FIG. 2 presents data for free enzymes with different blocking peptides.

FIG. 3 depicts data for separate enzyme-QD assembly with subsequent addition of different blocking peptides.

FIG. 4 provides data for mixed enzyme assembly on QD with subsequent addition of different blocking peptides.

FIGS. 5A-5C show data for positive, neutral, and negative blocking peptides, respectively.

FIGS. 6A-6C show data for enhancement with His9 and His12 peptides and fluorescence micrograph images denoting coacervate formation and localization of enzymes within the coacervates. In FIG. 6A one can see the normalized initial rate of NADP+ formation of the three enzyme cascade with no BP or with addition of the His9 and His12 peptides. The flux through the cascade is increased with the addition of the BP. FIG. 6B is a brightfield microscropy image of His9 peptides showing that they form coacervates. FIG. 6C is a fluorescence microscopy image of the same field as FIG. 6B showing that Alexa594 labeled enzymes are colocalized to the coacervate interior. Excitation was with a 562 nm laser and detection at 570-620 nm.

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of +10% of that stated.

### Overview

It was surprisingly found that conducting enzymatic cascade reactions in the presence of certain peptides (first intended as blocking peptides to prevent enzyme-crosslinking of QD) resulted in increased reaction rates. It is believed that these peptides operate by forming coacervates, which are membrane-less compartments where, in the case of coacervates formed by peptides, the liquid-liquid phase separation involves water as the continuous phase both inside and outside the coacervate. (see Abbas et al., Chem. Soc. Rev., 2021, 50, 3690-3705, incorporated herein by reference for the purposes of describing the formation and characterization of peptide-based coacervates).

### Examples

A three-enzyme cascade was used for these examples. The first enzyme in the cascade is Bacillus subtilis aspartate ammonia lyase (AspB), which catalyzes the conversion of fumaric acid to aspartic acid. The AspB gene utilized here encodes a 54.5 kDa monomer which assembles into a final active tetramer. The second enzyme is E. coli aspartokinase III (LysC) which phosphorylates aspartic acid in the second step using adenosine triphosphate (ATP) as the phosphate donor. The LysC gene encodes a 49.9 kDa monomer, which assembles into an active ˜100 kDa homodimer. The third enzyme in the cascade, E. coli aspartate-ß-semialdehyde dehydrogenase (Asd), catalyzes conversion of aspartyl phosphate to aspartate semialdehyde using nicotinamide adenine dinucleotide phosphate (NADPH) as the reducing co-factor. The Asd gene encodes a 41.4 kDa monomer which assembles into the active homodimer.

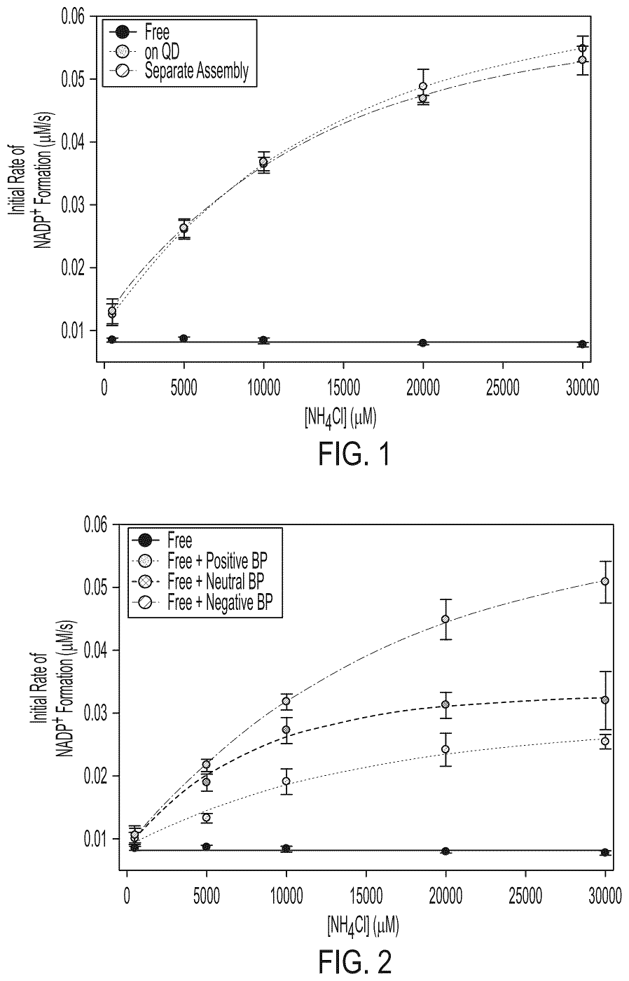

FIG. 1 shows results from a reaction to produce nicotinamide adenine dinucleotide phosphate (NADP+) without the use of added peptides. The reaction took place under the following conditions: 0.75 mM NADPH, 3 mM Fumaric Acid, 15 mM ATP, 17.5 mM MgCl₂ and variable NH₄Cl in 120 mM HEPES at pH 8 at 30 C. Enzyme concentrations were 60 nM AspB, 80 nM LysC, and 20 nM Asd. The concentration of QD was 30 nM (where present).

FIG. 2 provides data for free enzymes (not bound to QD) in the presence of different blocking peptides described in Table 1. The reaction took place under the following conditions: 0.75 mM NADPH, 3 mM Fumaric Acid, 15 mM ATP, 17.5 mM MgCl₂ and variable NH₄Cl in 120 mM HEPES at pH 8 at 30 C. Enzyme concentrations were 60 nM AspB, 80 nM LysC, and 20 nM Asd. The concentration of blocking peptide was 3000 nM (where present).

TABLE 1
Blocking Peptides.

| Positive | Neutral | Negative |
|---|---|---|
| JB434 | His-amine | BP-4R-2 |
| Ac-RaGGLA | [NH₂]GSWHHH | [NH₂]HHHHHH |
| (Aib)SGWKH6 | HHH[Amide] | GWDD[COOH] |
| (SEQ ID NO: 1) | (SEQ ID NO: 2) | (SEQ ID NO: 3) |

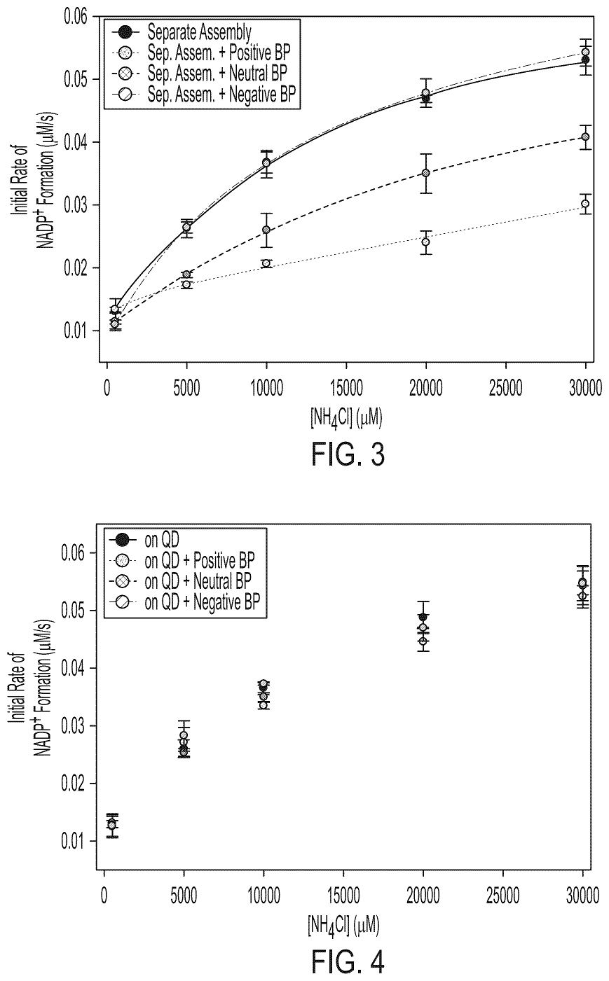

FIG. 3 depicts data for separate enzyme-QD assembly with subsequent addition of different blocking peptides. The reaction took place under the following conditions: 0.75 mM NADPH, 3 mM Fumaric Acid, 15 mM ATP, 17.5 mM MgCl₂ and variable NH₄Cl in 120 mM HEPES at pH 8 at 30 C. Enzyme concentrations were 60 nM AspB, 80 nM LysC, and 20 nM Asd. The concentration of QD was 30 nM and was present in all data. The concentration of blocking peptide was 3000 nM (where present).

FIG. 4 provides data for mixed enzyme assembly on QD with subsequent addition of different blocking peptides. The reaction took place under the following conditions: 0.75 mM NADPH, 3 mM Fumaric Acid, 15 mM ATP, 17.5 mM MgCl₂ and variable NH₄Cl in 120 mM HEPES at pH 8 at 30 C. Enzyme concentrations were 60 nM AspB, 80 nM LysC, and 20 nM Asd. The concentration of QD is 30 nM and is present in all data. The concentration of blocking peptide is 3000 nM (where present).

It was thought that peptide charge may relate to effectiveness. Table 2 below shows the charges of the enzymes at various pH levels.

TABLE 2
Enzymes.

| | MW of | Charge | Charge | Charge | Charge | Charge |
|---|---|---|---|---|---|---|
| Enzyme | Monomer | at pH 6 | at pH 7 | at pH 8 | at pH 9 | at pH 10 |
| AspB | 54.5 | −3.1 | −12.5 | −17.4 | −23.9 | −43 |
| LysC | 49.8 | −6.0 | −14.8 | −19.4 | −24.1 | −31.6 |
| Asd | 41.2 | 1.4 | −5.5 | −9.3 | −13.6 | −22.1 |

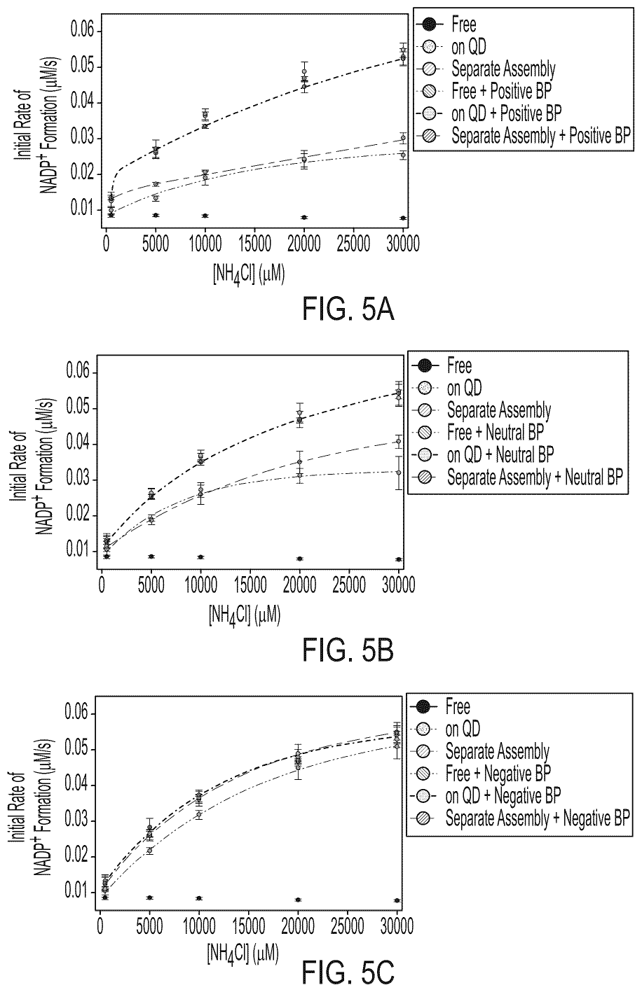

FIGS. 5A-5C show data for positive, neutral, and negative blocking peptides, respectively. The reactions took place under the following conditions: 0.75 mM NADPH, 3 mM Fumaric Acid, 15 mM ATP, 17.5 mM MgCl₂ and variable NH₄Cl in 120 mM HEPES at pH 8 at 30 C. Enzyme concentrations were 60 nM AspB, 80 nM LysC, and 20 nM Asd. The concentration of QD is 30 nM (where present). The concentration of blocking peptide is 3000 nM (where present).

Moreover, as detailed in the Appendix of Provisional Patent Application No. 63/482,369, the neutral peptide was also found to enhance activity of other enzymes.

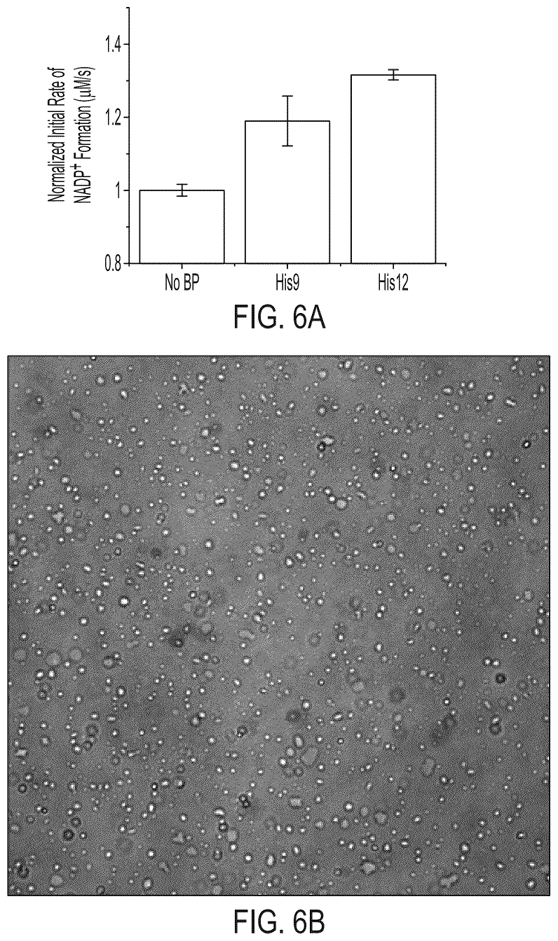

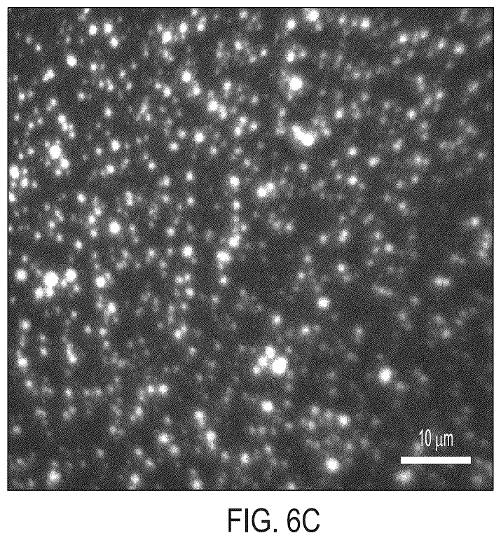

Also assayed were two peptides comprising primarily histidine, termed His9 and His12, with their sequences provided below in Table 3. FIGS. 6A to 6C show data relating to these peptides. FIG. 6A displays the normalized initial rate of NADP+ formation of the 3 enzyme cascade with no BP or with addition of the His9 and His12 peptides. The flux through the cascade is increased with the addition of the BP. FIG. 6B provides a brightfield microphotgraphic image of His9 peptides showing that they form coacervates. FIG. 6C is a fluorescence microscopy image of the same subject of FIG. 6B showing that Alexa594 labeled enzymes are colocalized to the coacervate interior.

TABLE 3
His Peptides.

| His9 | His12 |
|---|---|
| [NH₂]WHHHHH | [NH₂]WHHHHHH |
| HHHH[COOH] | HHHHHH[COOH] |
| (SEQ ID NO: 4) | (SEQ ID NO: 5) |

### Concluding Remarks

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention.

What is claimed is:

1. A method of conducting an enzyme cascade reaction, the method comprising:
providing a plurality of enzymes configured as an enzymatic cascade wherein the product of a first enzyme is the substrate of a second enzyme and so forth, wherein the enzymatic cascade comprises at least two different enzymes;
contacting the enzymatic cascade with a substrate of the first enzyme while contacting the enzymatic cascade with a peptide under conditions effective to form a peptide-driven coacervate; and
allowing a reaction to proceed so that each of the plurality of enzymes acts in succession to produce an end product,
wherein at least one of the enzymes comprises multiple polyhistidine tags acting to cross-link the nanoparticles into the cluster.

2. The method of claim 1 wherein said peptide is neither a product nor a substrate of any of the plurality of enzymes.

3. The method of claim 1, wherein the peptide is selected from the group consisting of SEQ ID NOs: 1 through 3, inclusive.

4. The method of claim 1, wherein the peptide is selected from the group consisting of SEQ ID NOs: 1 through 5, inclusive.

5. The method of claim 1 wherein the plurality of enzymes is bound to a plurality of nanoparticles, the nanoparticles being associated together as a cluster and wherein the nanoparticles in the cluster are closely associated with one another such that, on average, each nanoparticle is separated from the nearest neighboring nanoparticle by a distance of no more than about one nanoparticle diameter; and
wherein at least one of the enzymes comprises multiple polyhistidine tags acting to cross-link the nanoparticles into the cluster.

6. The method of claim 1 wherein the reaction is performed while minimizing stirring or mixing.

7. A method of conducting an enzyme cascade reaction, the method comprising:
providing a plurality of enzymes configured as an enzymatic cascade wherein the product of a first enzyme is the substrate of a second enzyme and so forth, wherein the enzymatic cascade comprises at least two different enzymes;
contacting the enzymatic cascade with a substrate of the first enzyme while contacting the enzymatic cascade with a peptide under conditions effective to form a peptide-driven coacervate; and
allowing a reaction to proceed so that each of the plurality of enzymes acts in succession to produce an end product,
wherein at least one of the enzymes comprises multiple polyhistidine tags acting to cross-link the nanoparticles into the cluster,
wherein the plurality of enzymes comprises Bacillus subtilis aspartate ammonia lyase (AspB), E. coli aspartokinase III (LysC), and E. coli aspartate-β-semialdehyde dehydrogenase (Asd).

8. The method of claim 7, wherein the peptide is selected from the group consisting of SEQ ID NOs: 1 through 3, inclusive.

9. The method of claim 7, wherein the peptide is selected from the group consisting of SEQ ID NOs: 1 through 5, inclusive.


SEQUENCE LISTING (5 sequences)

SEQ ID NO: 1 - protein, 14 aa, synthetic construct
GGLASGWKHHHHHH

SEQ ID NO: 2 - protein, 9 aa, synthetic construct
GSWHHHHHH

SEQ ID NO: 3 - protein, 10 aa, synthetic construct
HHHHHHGWDD

SEQ ID NO: 4 - protein, 10 aa, synthetic construct
WHHHHHHHHH

SEQ ID NO: 5 - protein, 13 aa, synthetic construct
WHHHHHHHHHHHH